(19) United States
(12) Patent Application Publication
(10) Pub. No.: US 2024/0177796 A1
(43) Pub. Date: May 30, 2024

(54) METHOD AND SYSTEM FOR GENERATING A PLURALITY OF ANTIBODY SEQUENCES

(71) Applicant: Innoplexus AG, Eschborn (DE)

(72) Inventors: Sudhanshu Kumar, Bokaro (IN); Joel Joseph, Palayi (IN); Ansh Gupta, Menhdawal (IN)

(73) Assignee: Innoplexus AG, Eschborn (DE)

(21) Appl. No.: 18/060,218

(22) Filed: Nov. 30, 2022

(51) Int. Cl. G16B 5/00; G06N 3/08; G16B 15/00

(52) U.S. Cl. CPC G16B 5/00; G06N 3/08; G16B 15/00

(57) ABSTRACT

A method and system for generating a plurality of new antibody sequences corresponding to a target from a single lead antibody sequence. A model is pre-trained with a training dataset of plurality of known antibody sequences to learn a structural pattern from the plurality of known antibody sequences. The method includes receiving the single lead antibody sequence. The lead antibody sequence has one or more regions. The regions have one or more lead framework regions (FR) and one or more lead complementarity determining regions (CDR). The pre-trained model is configured to process the single lead antibody sequence to identify a relationship between the one or more lead framework regions (FR) and one or more lead complementarity determining regions (CDR). A plurality of new antibody sequences are generated from the single lead antibody amino acid of the sequence by the pre-trained model based on the identified relationship and the structural pattern.

### FIELD OF TECHNOLOGY

Certain embodiments of the disclosure relate to generating a plurality of antibody sequences corresponding to an antigen target. More specifically, certain embodiments of the disclosure relate to method and system for generating a plurality of new antibody sequences of corresponding to a target from a single lead antibody sequence.

### BACKGROUND

Protein engineering is the process of developing useful or valuable proteins which have certain biological activities. An antibody is a protein component of the immune system that circulates in the blood, recognizes foreign substances, usually other protein structures called Antigens.

With the development of biomolecule development in the laboratory setting, techniques have been developed to generate artificial antibodies that can deal with specific antigens and also measure their activity towards those antigens. In-vitro antibody development is a very time-consuming process, as the possible combinations of Amino Acids to generate proteins are in the thousands of trillions and scientists often apply heuristics and homological modeling to come up with proteins that are similar to natural proteins.

In recent years, with the development of deep learning techniques and in-silico docking techniques, attempts have been made to do the complete protein generation process in-silico. Part of this attempt involves using natural language generation methods and text classification methods on protein and ligand sequences. However, known deep learning techniques are unable to generate sequences from a single training data.

Further limitations and disadvantages of conventional and traditional approaches will become apparent to one of skill in the art, through comparison of such systems with some aspects of the present disclosure as set forth in the remainder of the present application with reference to the drawings.

### SUMMARY

The aspects of the disclosed embodiments are directed to generating a plurality of new antibody sequences from a single lead antibody sequence corresponding to an input target.

Another aspect of the disclosed embodiments is to pre-train a model to generate a plurality of new antibody sequences corresponding to an input target.

A further aspect of the disclosed embodiments is to generate a plurality of new antibody sequences by a pre-trained model based on identified relationship between the one or more lead framework regions (FR) and one or more lead complementarity determining regions (CDR).

Yet another aspect of the disclosed embodiments is for the model to learn a structural pattern from the plurality of known antibody sequences corresponding to plurality of targets.

Another aspect of the disclosed embodiments is to implement a transfer learning methodology for generating a plurality of new antibody sequences from a single lead antibody sequence corresponding to the input target.

Another aspect of the disclosed embodiments is to select one or more of new antibody sequences from the generated plurality of new antibody sequences with high binding affinity.

A method is disclosed for generating a plurality of new antibody sequences corresponding to a target from a single lead antibody sequence, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram that illustrates an exemplary system for generating a plurality of new antibody sequences corresponding to a target from a single lead antibody sequence, in accordance with an exemplary embodiment of the disclosure.

FIG. 2 depicts a flowchart for generating a training dataset, in accordance with an exemplary embodiment of the disclosure.

FIG. 3 depict flowchart illustrating exemplary operations for generating a plurality of new antibody sequences corresponding to a target from a single lead antibody sequence, in accordance with various exemplary embodiments of the disclosure.

### DETAILED DESCRIPTION OF THE DISCLOSURE

Certain embodiments of the disclosure relate to generating a plurality of new antibody sequences corresponding to a target from a single lead antibody sequence. In the context of the current invention, a plurality of new antibody sequences is generated by a pre-trained model based on a relationship of lead antibody sequence between the one or more lead framework regions (FR) and one or more lead complementarity determining regions (CDR). The lead antibody sequence comprises of one or more regions, wherein the one or more region comprises of one or more lead framework regions (FR) and one or more lead complementarity determining regions (CDR).

Throughout this disclosure, the term “antibody sequence” refers to a protein sequence that defines the sequence of amino acids of an antibody. Preferably, the term antibody sequence, as disclosed in this specification, is always described as associated with a corresponding target. Further, the term “lead antibody sequence” corresponding to a target refers to a single antibody sequence known to have an effective binding affinity and therapeutic effect against the said target. It is reiterated herein that the term “lead antibody sequence” is always used in conjunction with a corresponding target. Furthermore, the term “known antibody sequences” refers to a plurality of antibody sequences which are known to be associated with at least one target and have demonstrated a high binding affinity with said target.

In accordance with various embodiments of the disclosure, a method for generating a plurality of new antibody sequences corresponding to a target from a single lead antibody sequence. The method comprises receiving, by one or more processors, the single lead antibody sequence, wherein the lead antibody sequence comprises of one or more regions, and wherein the one or more region comprises of one or more lead framework regions (FR) and one or more lead complementarity determining regions (CDR). The method further comprises processing, by one or more processors, the lead antibody sequence to identify a relationship between the one or more lead framework regions (FR) and one or more lead complementarity determining regions (CDR), wherein the lead antibody sequence is processed by a pre-trained model. Further, the method comprises generating, by one and more processors, the plurality of new antibody sequences by the pre-trained model based on the identified relationship and the structural pattern.

In accordance with an embodiment, the method comprises pre-processing a plurality of known antibody sequences corresponding to a plurality of targets to generate a training dataset.

In accordance with an embodiment, the method comprises processing the training dataset by a model to learn a structural pattern from the plurality of known antibody sequences, wherein the structural pattern comprises a set of biological and chemical rules that define a basic structure for each of the plurality of known antibody sequences.

In accordance with the preferred embodiment, the pre-trained model is configured to generate the plurality of new antibody sequences from the single lead antibody amino acid sequence based on transfer learning, wherein transfer learning reuses the learned structural pattern as a starting point for generating the plurality of new antibody sequences from the single lead antibody sequence.

In accordance with an embodiment, the model comprises one of Markov Chain model, Long Short-Term Memory neural networks, GPT-2, and ARCNN to generate the plurality of amino acid sequences based on the transfer learning.

In accordance with an embodiment, the method comprises selecting one or more of the generated plurality of new antibody sequences with high relevance or binding affinity.

In accordance with another aspect of the disclosure, a system for generating a plurality of new antibody sequences corresponding to a target from a single lead antibody sequence is disclosed. The system comprises at least one server communicable coupled with at least one database. The server comprising one or more processors configured to receive the single lead antibody sequence, process the lead amino acid sequence to identify relationship between the one or more lead framework regions (FR) and one or more lead complementarity determining regions (CDR), wherein the lead amino acid sequence is processed by a model, and generate the plurality of amino acid sequences by the model based on the identified relationship and structural pattern, wherein the model is pretrained based on a plurality of known antibody sequences thereby learning a structural pattern for antibody sequences.

In accordance with an embodiment, the at least one server is configured to pre-process a plurality of known antibody sequences to generate a training dataset.

In accordance with an embodiment, the at least one server is configured to process the training dataset by the model to learn a structural pattern of the plurality of known antibody sequences corresponding to a plurality of target, wherein the structural pattern comprises a set of biological and chemical rules that define a basic structure of each of the plurality of known antibody sequences.

In accordance with another aspect of the disclosure, wherein the pre-trained model is configured to generate the plurality of amino acid sequences from the single lead amino acid of the sequence based on transfer learning, wherein the model reuses the learned structural pattern as a starting point for generating the plurality of amino acid sequences from the single lead amino acid.

In accordance with an embodiment, the model comprises one of Markov Chain model, Long Short-Term Memory) neural networks, GPT-2, and ARCNN.

In accordance with an embodiment, the at least one server is configured to select one or more of the generated plurality of new antibody sequences with high relevance and/or binding affinity.

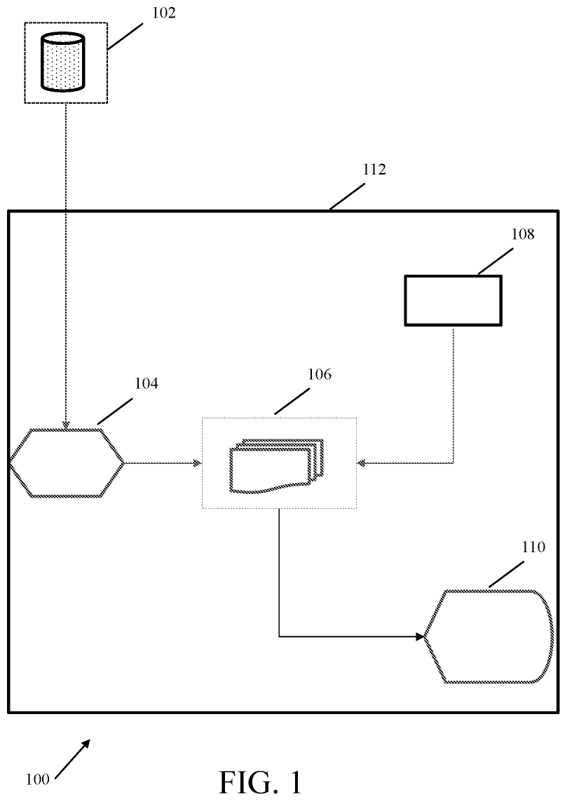

FIG. 1 is a block diagram that illustrates an exemplary system for generating a plurality of antibody sequences f corresponding to a target from a single lead antibody sequence. Referring to FIG. 1, a system 100 includes at least one server 112 and at least one database arrangement 102. The at least one server 112 comprises of a pre-processing module 104, a training and generation module 106, an input module 108, and an output module 110. The at least one server 112, the database arrangement 102 is communicably coupled via the communication network (not shown).

In an embodiment, a single lead antibody sequence of a target is received to generate a plurality of new antibody sequences corresponding to the target. In an embodiment, an antibody generation model is trained with available and known antibody sequences data corresponding to different antigens so that the model learns to generate the structural pattern of binding protein sequences across antigen targets. Based on the learnt structural patters, CDR regions are generated by the model keeping the FR regions as prefixes. Beneficially, a large library of candidate sequences for binding is generated by the model.

In an embodiment, transfer learning is implemented for generation of plurality of antibody sequences. Since, the model has received sequences from different targets to train and thus the model can learn from sequences a variety of targets and generate new sequences for a particular target. In an embodiment, the model can perform well even if there is inadequate training data for that particular target for generation of sequences.

The deep learning based models are trained on antibody-antigen complexes with corresponding binding affinity values so as to generate the data. In an embodiment, sequences from a variety of target landscapes are considered. The length of sequences from each target is different and even FWR and CDR fragments are also different. The model is able to learn from the sequences coming from different targets and when we provide some prefix from our lead sequence, it generates sufficiently good sequences for that particular target also.

The at least one server 112 further comprises a memory, a storage device, an input/output (I/O) device, a user interface, and a wireless transceiver. The at least one database 102 is external or remote resources but communicatively coupled to the at least one server 112 via a communication network.

In some embodiment of the disclosure, the pre-processing module 104, the training and generation module 106, the input module 108, and the output module 110 are integrated with other processors and modules to form an integrated system. In some embodiments of the disclosure the one or more processors of the at least one server 112 may be integrated in any order and other combination modules to form an integrated system. In some embodiments of the disclosure, as shown, the pre-processing module 104, the training and generation module 106, the input module 108, and the output module 110 may be distinct from each other. Other separation and/or combination of the various processing engines and entities of the exemplary system 100 illustrated in FIG. 1 may be done without departing from the spirit and scope of the various embodiments of the disclosure.

The at least one database 102 is configured to store the plurality of known antibody sequences. The database may be capable of providing mass storage to the at least one server 112. In some embodiments, the database may be or contain a computer-readable medium, such as a hard disk device, an optical disk device, or a tape device, a flash memory or other similar solid state memory device, or an array of devices, including devices in a storage area network or other configurations. A computer program product may be tangibly embodied in an information carrier. The information carrier may be a computer-readable or machine-readable medium, such as database. The computer program product may also contain instructions that, when executed, perform one or more methods, such as those described in the disclosure. In an embodiment, the plurality of known antibody sequence has high binding affinity value.

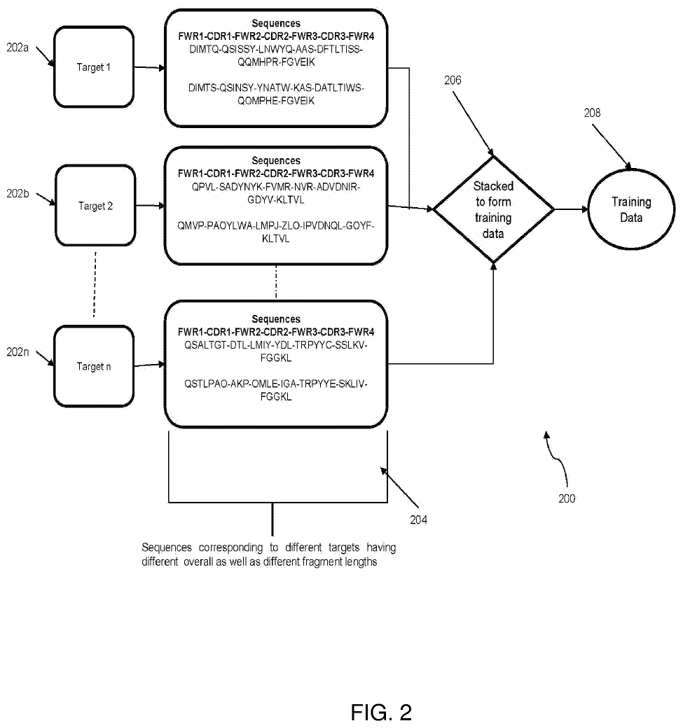

The pre-processing module 104 comprises of suitable libraries, logic, and/or code that may be operable to pre-process the plurality of known antibody sequence of the target in conjunction with the one or more processors. More specifically, the pre-processing module 104, in conjunction with the one or more processors, may enable the at least one server 112 to generate a training dataset suitable of training the training and generation module 106. In an embodiment, the pre-processing module 104 receives the plurality of known antibody sequence corresponding to a plurality of target from the database arrangement 102. Referring to FIG. 2, a flowchart is depicted for generating a training dataset, wherein the training dataset is generated post pre-processing the plurality of known antibody sequences. In an embodiment, the pre-processing module 104 receives the plurality of known antibody sequences from the at least one database 102. In an embodiment, the pre-processing module 104 receives a plurality of target, Target 1 202a, Target 2 202b, . . . Target n 202n. In an embodiment, there are n number of sequences 204 for each target. In an embodiment, the sequences 204 are stacked 206 to generate target data 208.

In an embodiment, the pre-processing module 104 is configured to process the received plurality of known antibody sequences corresponding to the plurality of targets to identify one or more regions in the received plurality of known antibody sequences. The one or more regions in the received plurality of known antibody sequences is identified by the pre-processing module 104 using one or more standard bio-informatic algorithms. The one or more regions comprises of one or more known framework regions (FR) and one or more known complementarity determining regions (CDR). In an embodiment, the pre-processing module 104 is configured to insert spaces between the characters to convert the characters into information capable of being interpreted contextually by the training and generation module 106. In an embodiment, the spaced characters are identified as amino acids by the training and generation module 106 to train the model.

The training and generation module 106 comprises suitable libraries, logic, and/or code that may be operable to implement the training and generation function in conjunction with the one or more processors. More specifically, the training and generation function, in conjunction with the one or more processors, may enable the at least one server 112 to generate a plurality of antibody sequences corresponding to the targets from the single lead antibody sequence. In an embodiment, the training and generation module 106 is configured to receive the single lead antibody sequence and process the single lead antibody sequence to identify a relationship between the one or more lead framework regions (FR) and one or more lead complementarity determining regions (CDR). In an embodiment, the training and generation module 106 is configured to implement a pre-trained model to identify the relationship between the one or more lead framework regions (FR) and one or more lead complementarity determining regions (CDR). Further, the training and generation module 106 is configured to generate the plurality of new antibody sequences based on the identified relationship. In an embodiment, the pre-trained model is operable to generate the plurality of amino acid sequences based on the identified relationship. In an embodiment, the model comprises one of Autoregressive Convolutional Neural Network, Long Short-Term Memory (LSTM) networks, Markov model, and GPT-2 model. The inventor expects skilled artisans to employ one or more model and its variations as appropriate. The models mentioned herein should not be considered limiting, and the inventors intend for the present invention to be practiced otherwise than specifically described herein. In an embodiment, the training and generation module 106 is configured to receive the training dataset from the pre-processing module 104 to train the model. In an embodiment, the model is configured to learn a structural pattern of the plurality of known antibody sequences from the training dataset. The structural pattern comprises a set of biological and chemical rules that define a basic structure of each of the plurality of known antibody sequences.

In an embodiment, the model is trained on the training dataset to identify structural pattern of the plurality of known antibody sequences. It is generally known in the art that the plurality of known antibody sequences corresponding to the plurality of targets have a basic structure. In an embodiment, the model is operable to identify the basic structure of each of the plurality of known antibody sequences. Beneficially, the basic structure of each of the plurality of antibody sequence comprises of a set of biological and chemical rules. Beneficially, training the model on the training dataset prevents it from learning everything from scratch, instead, it transfers and reuses the old knowledge (i.e., the structural pattern of plurality of known antibody sequences) of what it has learned in the past to understand new knowledge (i.e., the identified relationship of the single lead antibody sequence) and handle new tasks. In an embodiment, the training and generation model 106 pre-trains the model using the structural pattern of plurality of known antibody sequences before the generation function is of the antibody sequences is initialized. In an embodiment, the training and generation module 106 receives the single lead antibody sequence. The pre-trained model processes the single lead antibody sequence to identify the relationship between the one or more lead framework regions (FR) and one or more lead complementarity determining regions (CDR). Subsequently, the training and generation module 106 uses the structural pattern of plurality of known antibody sequences (old knowledge) and the identified relationship (new knowledge) and helps the pre-trained model successfully perform the generation of the new antibody sequences from the learned structural pattern and identified relationship.

In an embodiment, the training and generation module 106 is operable to implement the pre-trained model to generate the plurality of new antibody sequences from the single lead antibody amino acid sequence based on the transfer learning. The transfer learning methodology reuses the learned structural pattern as a starting point for generating the plurality of new antibody sequences from the single lead antibody sequence. In an embodiment, the training and generation module 106 is configured to operate the pre-trained model to generate the plurality of new antibody sequences based on the identified relationship and the learned structural pattern. In an embodiment, the pre-trained model is configured to use transfer learning methodology to reuse the structural pattern as a starting point and use the identified relationship to generate the plurality of new antibody sequences. In an embodiment, autoregressive (especially deep learning) models learn to identify intrinsic structures and patterns in the data. With multiple iterations of computation going through multiple layers (deep) they optimize themselves to create multiple levels of abstraction to represent data. The data is generally represented in higher dimensions to give them a meaning with respect to relations they may hold with other parts of the data.

In an embodiment, the training and generation module 106 is operable to select one or more of the plurality of new antibody sequences with high relevance and/or binding affinity. The training and generation module 106 is operable to consider the frequency of generation of sequences, and if a sequence is generated multiple times, it is more probable that it would be a sequence with better binding affinity with the target.

In an embodiment, the Markov Chain model a stochastic model, i.e., the model is based on random probability distribution. Markov Chain models the future state i.e., in case of text generation, the next word, based on the previous state (i.e., previous word or sequence). The model is memory-less—the prediction depends only on the current state of the variable (it forgets the past states; it is independent of preceding states). On the other hand, it's simple, fast to execute and light on memory.

In an embodiment, using Markov Chain model for text generation requires the following steps:
a. Load the dataset and preprocess text.
b. Extract from text the sequences of length n (current state) and the next words (future state).
c. Build the transition matrix with the probability values of state transitions.

In order to build a transition matrix, the Markov chain model processes the entire text and counts all transitions from a particular sequence (ngram) to the next word. The values are then stored in a matrix, where rows correspond to the particular sequence and columns to the particular token (next word). The values represent the number of occurrences of each token after the particular sequence. Since the transition matrix should contain probabilities, not counts, in the end the occurrences are recalculated into probabilities. The matrix is saved in scipy.sparse format to limit the space it takes up in the memory. Thereafter the next word is predicted based on the probability distribution for state transition.

In an embodiment, Long Short-Term Memory neural networks used in classification, translation and text generation. The Long Short-Term Memory neural networks generalize across sequences rather than learn individual patterns, which makes the model a suitable tool for modeling sequential data. In order to generate text, they learn how to predict the next word based on the input sequence. The step-by-step approach for text Generation with LSTM:
a) Load the dataset and preprocess text. Extract sequences of length n (X, input vector) and the next words (y, label).
b) Build DataGenerator that returns batches of data.
c) Define the LSTM model and train it.
d) Predict the next word based on the sequence.

In an embodiment, Open AI GPT-2 is used which is a transformer-based, autoregressive language model that shows competitive performance on multiple language tasks, especially (long form) text generation. GPT-2 is trained on 40 GB of high-quality content using the simple task of predicting the next word. The model does it by using attention. It allows the model to focus on the words that are relevant to predicting the next word. Hugging Face Transformers library provides everything you need to train/fine-tune/use transformers models. The model is implemented as follows:
a) Load Tokenizer and Data Collator
b) Load data and create a Dataset object
c) Load the Model
d) Load and setup the Trainer and Training Arguments
e) Fine-tune the model
f) Generate text with the Pipeline

In an embodiment, an autoregressive CNN based sequence generation model uses 1 dimensional CNNs on the one hot encoding matrix of the sequence. During training, its input is the masked training sequence and expected output is the unmasked sequence. It uses reconstruction loss. During inference, input is the prefix added with masks and output is the prefix added with the generated fragment of the sequence.

The input module 108 is operable in conjunction with the training and generation module 106. The input module 108 comprises of suitable libraries, logic, and/or code that may be operable to receive the single lead antibody sequence against an input target. In an embodiment, the input module 108 operable to manually receive, from a user, the single lead antibody sequence.

The output module 110 comprise suitable logic, circuitry, and interfaces that may be configured to present the results i.e., generated plurality of new antibody sequences of the input target. The results are presented in form of an audible, visual, tactile, or other output to the user, such as a researcher, a scientist, a principal investigator, data manager, and a health authority, associated with the at least one server 112. As such, the user interface may include, for example, a display, one or more switches, buttons, or keys (e.g., a keyboard or other function buttons), a mouse, and/or other input/output mechanisms. In an example embodiment, the user interface may include a plurality of lights, a display, a speaker, a microphone, and/or the like. In some embodiments, the user interface may also provide interface mechanisms that are generated on the display for facilitating user interaction. Thus, for example, the user interface may be configured to provide interface consoles, web pages, web portals, drop down menus, buttons, and/or the like, and components thereof to facilitate user interaction.

The communication network may be any kind of network, or a combination of various networks, and it is shown illustrating exemplary communication that may occur between the at least one database 102 and the at least one server 112. For example, the communication network may comprise one or more of a cable television network, the Internet, a satellite communication network, or a group of interconnected networks (for example, Wide Area Networks or WANs), such as the World Wide Web. Accordingly, other exemplary modes may comprise uni-directional or bi-directional distribution, such as packet-radio, and satellite networks.

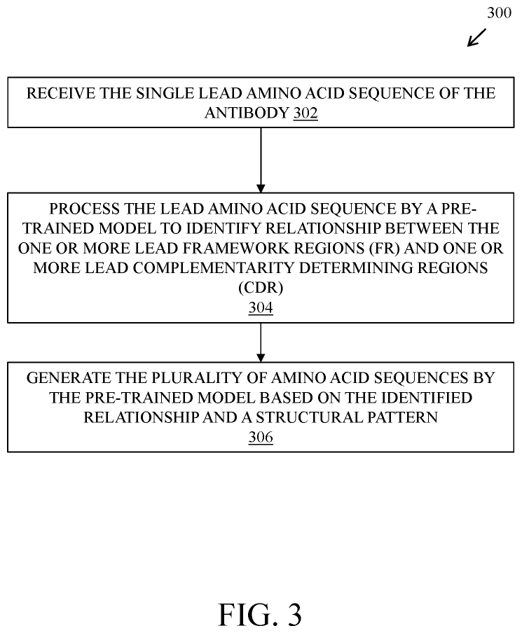

FIG. 3 depicts a flowchart illustrating exemplary operations for generating a plurality of new antibody sequences corresponding to a target from a single lead antibody sequence. Flowcharts 300 is described in conjunction with FIG. 1

At step 302, the single lead antibody sequence is received, wherein the lead antibody sequence consists of one or more regions, wherein the one or more region comprises of one or more lead framework regions (FR) and one or more lead complementarity determining regions (CDR).

At step 304, the lead antibody sequence is processed by a pre-trained model to identify relationship between the one or more lead framework regions (FR) and one or more lead complementarity determining regions (CDR).

At step 306, the plurality of new antibody sequences are generated by the pre-trained model based on the identified relationship and a structural pattern.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and/or code (if any is necessary) to perform the function, regardless of whether performance of the function is disabled, or not enabled, by some user-configurable setting.

The terminology used herein is for the purpose of describing particular embodiments only and is not intended to be limiting of embodiments of the disclosure. As used herein, the singular forms “a”, “an” and “the” are intended to include the plural forms as well, unless the context clearly indicates otherwise. It will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any non-transitory form of computer readable storage medium having stored therein a corresponding set of computer instructions that upon execution would cause an associated processor to perform the functionality described herein. Thus, the various aspects of the disclosure may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

Another embodiment of the disclosure may provide a non-transitory machine and/or computer-readable storage and/or media, having stored thereon, a machine code and/or a computer program having at least one code section executable by a machine and/or a computer, thereby causing the machine and/or computer to perform the steps as described herein for determining combination drug and use in pancreatic cancer treatment.

The present disclosure may also be embedded in a computer program product, which comprises all the features enabling the implementation of the methods described herein, and which when loaded in a computer system is able to carry out these methods. Computer program in the present context means any expression, in any language, code or notation, either statically or dynamically defined, of a set of instructions intended to cause a system having an information processing capability to perform a particular function either directly or after either or both of the following: a) conversion to another language, code or notation; b) reproduction in a different material form.

Further, those of skill in the art will appreciate that the various illustrative logical blocks, modules, circuits, algorithms, and/or steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The methods, sequences and/or algorithms described in connection with the embodiments disclosed herein may be embodied directly in firmware, hardware, in a software module executed by a processor, or in a combination thereof. A software module may reside in RAM memory, flash memory, ROM memory, EPROM memory, EEPROM memory, registers, hard disk, physical and/or virtual disk, a removable disk, a CD-ROM, virtualized system or device such as a virtual server or container, or any other form of storage medium known in the art. An exemplary storage medium is communicatively coupled to the processor (including logic/code executing in the processor) such that the processor can read information from, and write information to, the storage medium. In the alternative, the storage medium may be integral to the processor.

While the present disclosure has been described with reference to certain embodiments, it will be noted understood by, for example, those skilled in the art that various changes and modifications could be made and equivalents may be substituted without departing from the scope of the present disclosure as defined, for example, in the appended claims. In addition, many modifications may be made to adapt a particular situation or material to the teachings of the present disclosure without departing from its scope. The functions, steps and/or actions of the method claims in accordance with the embodiments of the disclosure described herein need not be performed in any particular order. Furthermore, although elements of the disclosure may be described or claimed in the singular, the plural is contemplated unless limitation to the singular is explicitly stated. Therefore, it is intended that the present disclosure is not limited to the particular embodiment disclosed, but that the present disclosure will include all embodiments falling within the scope of the appended claims.

What is claimed is:

1. A method for generating a plurality of new antibody sequences corresponding to an input target from a single lead antibody sequence, comprising:
Receiving the single lead antibody sequence, wherein the lead antibody sequence comprises one or more regions, and wherein the one or more region comprises one or more lead framework regions (FR) and one or more lead complementarity determining regions (CDR),
Processing the single lead antibody sequence to identify a relationship between the one or more lead framework regions (FR) and one or more lead complementarity determining regions (CDR), wherein the lead antibody sequence is processed by a pre-trained model, and
Generating the plurality of new antibody sequences by the pre-trained model based on the identified relationship and a structural pattern.

2. The method of claim 1, wherein the method comprises pre-processing a plurality of known antibody sequences to generate a training dataset.

3. The method of claim 2, wherein the method comprises processing the training dataset by the model to learn a structural pattern of the plurality of known antibody sequences, wherein the structural pattern comprises set of biological and chemical rules that define a basic structure of each of the plurality of known antibody sequences.

4. The method claim 1, wherein the model is configured to generate the plurality of new antibody sequences from the single lead antibody amino acid sequence based on the transfer learning, wherein the transfer learning reuses the learned structural pattern as a starting point for generating the plurality of new antibody sequences from the single lead antibody sequence.

5. The method claim 4, wherein the model comprises one of Markov Chain model, Long Short-Term Memory) neural networks, GPT-2, and ARCNN.

6. The method of claim 1, wherein the method comprises selecting one or more of the generated plurality of new antibody sequences with high relevance and/or binding affinity.

7. A system for generating a plurality of new antibody sequences corresponding to a target from a single lead antibody sequence, wherein the system comprises:
at least one server communicable coupled with at least one database, wherein the at least one server comprises of one or more processors configured to
receive the single lead antibody sequence, wherein the lead antibody sequence comprises of one or more regions, and wherein the one or more region comprises of one or more lead framework regions (FR) and one or more lead complementarity determining regions (CDR),
process the lead antibody sequence to identify a relationship between the one or more lead framework regions (FR) and one or more lead complementarity determining regions (CDR), wherein the lead antibody sequence is processed by a pre-trained model, and
generate the plurality of amino acid sequences by the pre-trained model based on the identified relationship and a structural pattern.

8. The system as claimed in claim 7, wherein the at least one server is configured to pre-process a plurality of known antibody sequences to generate a training dataset.

9. The system as claimed in claim 8, wherein the at least one server is configured to process the training dataset by the pre-trained model to learn a structural pattern of the plurality of known antibody sequences, wherein the structural pattern comprises set of biological and chemical rules that define the structure of the plurality of known antibody sequences.

10. The system as claimed in claim 7, wherein the pre-trained model is configured to generate the plurality of new antibody sequences from the single lead antibody amino acid sequence based on the transfer learning methodology, wherein the transfer learning methodology reuses the learned structural pattern as a starting point for generating the plurality of new antibody sequences from the single lead antibody sequence.

11. The system as claimed in claim 10, wherein the pre-trained model comprises one of Markov Chain model, Long Short-Term Memory) neural networks, GPT-2, and ARCNN.

12. The system as claimed in claim 7, wherein the at least one server is configured to select one or more of the generated plurality of new antibody sequences with high relevance and/or binding affinity.
